(19) United States
(12) Patent Application Publication
(10) Pub. No.: US 2017/0035029 A1
(43) Pub. Date: Feb. 9, 2017

(54) Systems and Methods for Data Driven Optimization of Dog Training

(71) Applicant: Radio Systems Corporation, Knoxville, TN (US)

(72) Inventor: Christopher E. Mainini, Knoxville, TN (US)

(21) Appl. No.: 14/842,512

(22) Filed: Sep. 1, 2015

Related U.S. Application Data
(60) Provisional application No. 62/201,666, filed on Aug. 6, 2015.

(51) Int. Cl. A01K 29/00; A01K 15/02

(52) U.S. Cl. CPC A01K 29/00; A01K 15/025

(57) ABSTRACT

A method of developing a dog is described. The method comprises testing a dog to determine a personality profile, and further testing the dog to determine a cognitive skill level. The method also comprises (i) identifying an optimum training protocol for the dog, (ii) identifying an optimum developmental product for the dog, (iii) identifying an optimum pet toy for the dog, or (iv) combinations thereof, based upon a combination of the determined personality profile and the determined cognitive skill level. In one aspect, evaluation of intelligence and personality are part of a single test. A system for training a dog is also provided. The system includes a test for determining a profile of the dog. The system also includes a products database and a training database. The databases are developed by identifying optimum training products and training protocols based on dog profiles.

### CROSS REFERENCE TO RELATED APPLICATIONS

This application claims the benefit of U.S. Provisional Patent Application No. 62/201,666 filed Aug. 6, 2015. That application is entitled “Systems and Methods for Data Drive Optimization of Dog Training,” and is incorporated herein by reference in its entirety.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### BACKGROUND OF THE INVENTION

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

### FIELD OF THE INVENTION

The present disclosure relates to the field of animal training. More specifically, the present invention relates to methods for training a canine based upon evaluations of the dog's personality and its intelligence. The application also relates to the selection of appropriate toys and training devices based upon such evaluations.

### DISCUSSION OF TECHNOLOGY

It is known that dogs, like people, have different levels of intelligence. For canines, intelligence is a combination of breed and training. Intelligence is determined by the ability of the dog to learn, which generally means the ability to associate sounds or motions (sometimes referred to as “somatosensory feedback or biofeedback”) with objects or actions.

The border collie (or Scotch sheep dog) is often considered to be the brightest dog. Other breeds that are considered to have higher cognitive skill levels include the golden retriever, the Labrador retriever, the Shetland sheepdog, the German shepherd, and the Doberman pinscher.

Studies into the thought processes and personality types of canines have recently been conducted by Professor Brian Hare at Duke University. Dr. Hare is the Director of the Duke Canine Cognition Center. Dr. Hare has determined that, compared to people, most dogs are considered to have the cognition of the average toddler. However, some dogs, such as border collies, are known (or at least widely believed) to have higher levels of conceptual learning.

Professor John W. Pilley of Wofford College recently authored a book about his dog Chaser. The title of the book is “Chaser: Unlocking the Genius of the Dog Who Knows a Thousand Words,” (Houghton Mifflin Harcourt). See, e.g., “Meet the Dog Who Knows 1,000 Words,” (Time Magazine Online, Nov. 5, 2013, http://ideas.time.com/2013/11/05/your-dog-is-toddler). Chaser is an example of a highly trained border collie.

In contrast to border collies, some breeds are considered (fairly or unfairly) to have lower cognitive skill levels. These generally include the so-called hounds including the Afghan hound, the Basset hound, the Beagle and the bloodhound. Some have also placed the bulldog, the pug, the Chow Chow, the mastiff and the Pekingese in this lower category, although there are undoubtedly exceptions within each breed and individual dog owners may rightfully have strong feelings about their individual dog or their experiences with canine pets.

Criteria for determining the intelligence of a dog generally include sense of direction, memory, ability to be trained to follow basic commands (such as “sit” or “stay”), ability to adapt to new surroundings, and ability to navigate an obstacle course. Based on this criteria, one might expect that a dog who is more intelligent is more easily trained than a dog that has lower cognitive skills.

Dr. Hare has developed an on-line assessment for a dog. His website is found at www.dognition.com, and includes the heading “Is Your Dog a Genius?” A review of Dr. Hare's website reveals that the protocol for determining a dog's developmental level results in a dog being assigned to one of the following profiles: Ace, Charmer, Socialite, Expert, Renaissance Dog, ProtoDog, Einstein, Maverick and Stargazer. For example, the Charmer is described as follows:
Charmers have exceptional social skills, meaning they can read human body language like a book. Seeing as these social skills are paired with just the right amount of independent problem solving skills, it's no surprise that Charmers can be quite mischievous! Indeed, this combination of cognitive skills sets means that many Charmers are not above using their owner's social information to get their own way. Whether cooperative or sneaky, these dogs are deeply in tune with their owner and very clever.
   https://www.dognition.com/profiles/charmer. (It is expected that Dr. Hare and his co-founders at Canines, Inc. have copyright protection on various aspects of the Dognition assessment protocol, including possibly the naming scheme, the software behind the website, artwork associated with the naming scheme, and the text of the website. Accordingly, Applicant seeks to use no more of this material herein than is minimally necessary to provide an appropriate patent application Background, and full attribution is given to Canines, Inc. of North Carolina.)

It has been suggested that just as different dogs have different cognitive skill levels, or abilities to learn, different dogs also have different personality types. U.S. Patent Publ. No. 2011/01396086 entitled “Systems and Methods for Generating Personality Profiles for Animals” disclosed the idea of generating a profile of animals in order to determine compatibility. Dogs were assigned one of the following categories for different personality traits: orange, gold, blue or green. A matrix was offered at FIG. 4 of the published application, which uses a scoring system to identify personality type for a Canine. FIG. 4 of U.S. Patent Publ. No. 2011/01396086 is somewhat duplicated herein as FIG. 3.

A need exists to improve upon the manner in which dogs are trained based upon cognitive skills and personality characteristics. Further, a need exists for identifying ways in which dogs are best entertained or taught using toys that are selected based upon intelligence, personality characteristics, or a combination thereof. Regarding training devices and developmental products, a need exists for a smarter and more efficient dog training device that can provide various types and levels of stimuli to the dog, both positive and negative.

### SUMMARY OF THE INVENTION

A method of developing a dog is first provided herein. The method first comprises testing the dog to determine its profile. The method then includes (i) identifying an optimum training protocol for the dog, (ii) identifying an optimum developmental product for the dog, (iii) identifying an optimum pet toy for the dog, or (iv) combinations thereof, based upon the determined profile.

The profile may be based upon a (i) personality of the dog, (ii) a cognitive skill level of the dog, or (iii) both. Accordingly, in one aspect, the method further includes testing the dog to determine a personality profile. The personality profile may comprise a matrix of personality traits which are scored to produce a primary spectrum. The personality traits are identified by a pet owner based upon (i) observations of the dog, (ii) empirical testing of the dog, (iii) breed type of the dog, (iv) living environment, or (v) combinations thereof.

In another aspect, the method further includes testing the dog to determine a cognitive skill level of the dog. Testing the dog to determine a cognitive skill level may comprise selecting an intelligence range from a plurality of intelligence ranges. The intelligence ranges are weighted to correspond to (i) breed type, (ii) degree of obedience training, or (iii) both, for the dog.

The method preferably further includes training the dog (i) based on the identified optimum training protocol, (ii) using the identified optimum developmental product, or (iii) both. Alternatively, the method further includes purchasing an optimum pet toy for the dog.

A system for training a dog is also provided herein. The system includes:
a personality test for identifying at least one personality type of a dog;
a cognitive skill test for identifying at least one intelligence level of the dog;
a products database including information of optimal training products for each of the at least one personality type and for each of the at least one intelligence level, wherein the optimal training product information associates a personality type combined with a cognitive skill level with one or more corresponding optimal training products; and
a training database including information of optimal training protocols for each of the at least one personality type and for each of the at least one intelligence level, wherein the optimal training protocol information associates a personality type combined with a cognitive skill level with one or more corresponding training protocols.

The personality test is derived from (i) observations of the dog, (ii) empirical testing of the dog while the dog performs exercises, (iii) assigning a breed type for the dog, (iv) living environment for the dog, or (v) combinations thereof. The intelligence test is derived from (i) considering breed type, (ii) considering degree of obedience training, or (iii) both, for the dog.

In one aspect, the personality test and the intelligence test are combined into a single test such that optimal training product information associates the assessed personality type combined with the assessed intelligence level, as part of a single test, with one or more corresponding optimal training products. Similarly, optimal training protocol information associates the assessed personality type combined with the assessed intelligence level with one or more corresponding training protocols. It is understood that with a dog, the domain of cognitive skill level and the domain of personality may overlap.

### BRIEF DESCRIPTION OF THE DRAWINGS

So that the manner in which the present application can be better understood, certain illustrations and figures are appended hereto. It is to be noted, however, that the drawings illustrate only selected embodiments and elements of the systems and methods described herein and are therefore not to be considered limiting in scope for the systems and methods as described herein may admit to other equally effective embodiments and applications.

FIG. 1 shows a method for optimizing pet training products and training protocols, in one embodiment.

FIG. 2 shows a method for optimizing pet training products and training protocols, in a further embodiment.

FIG. 3 is a matrix used for scoring a dog based upon personality traits. The matrix is derived from FIG. 4 of U.S. Patent Publ. No. 2011/01396086.

FIG. 4 is a flowchart showing a method of building two databases; a dog personality database of optimal training products for one or more of the personality types, and a dog intelligence database of optimal training protocols associated with one or more of the intelligence types, or levels.

FIG. 5 is a flowchart showing a method of administering dog personality and intelligence tests coordinated with associated personality and intelligence databases, thus determining optimum training products and protocols for the dog.

FIG. 6 is a flowchart showing a method of identifying combinations of optimum training protocol, pet toys, and developmental products based on personality profile and intelligence range. Furthermore, the method is shown whereby intelligence ranges are determined based on testing and a matrix of personality traits.

FIG. 7 is a diagram of a particular training and developmental product, a smart dog collar system.

### DETAILED DESCRIPTION

Historically, the exceptional ability of dogs to get along with people has set them aside from all others in the animal kingdom. Their unique abilities to “communicate” with humans has made them “man's best friend.” Recent advances in the research of dogs has identified areas of interest, including mental skills, spontaneous inferences and communicative intentions. In addition, research has shown that these, and other intelligence traits in dogs are individual pet specific traits. This means that dogs have varying degrees of skill in different areas.

Research continues to uncover details about the mental make-up of dogs. Opportunities arise to exploit these findings for the improvement of dog training methods. For example, researchers have learned more about the way dogs think in the last decade than ever before. There are a number of unique attributes related to dogs that are of interest to dog trainers. These attributes are also of interest to the developers of training protocols and the manufacturers of pet training products and toys.

Researchers have developed “tests” to rate pet skills Similar to an IQ test or a Myers-Briggs personality assessment, these tests provide insight into the inner workings of the dog's mind. It would seem reasonable to couple the test results with the development of personalized training protocols that lend themselves to optimized learning, thereby producing novel methods. Also, training product development teams may utilize this information to improve the development process.

As indicated above, each dog also has a certain intelligence. A dog's intelligence indicates an ability of an animal to learn a particular behavior or response. The higher the intelligence, the quicker an animal may learn the particular behavior or response. Intelligence type may suggest training methods and protocols, e.g. an owner may motivate/reward an extremely intelligent dog with treats that require the dog to determine a method/technique for extracting treats from an object.

As also indicated above, it is believed that each dog has a unique personality type. Efforts have been made to characterize personality types and to develop assessment tools for identifying the personality types. A personality assessment may identify different predominant behaviors and tendencies of an animal. For example, a dog may be predominantly sociable. Alternatively, a dog may be predominantly aggressive or predominantly submissive. One dog may enjoy (or at least tolerate) the sometimes abusive behavior of a small child, while another dog may shy away from such behavior or even react with hostility.

Differing personality types may suggest corresponding optimum training products. As just one example, if a dog exhibits a predominantly aggressive disposition, then the dog may require products that command the animal's firm attention. An example would be a collar device that applies a strong negative stimulus. However, a dog that exhibits a predominantly sociable disposition may respond to products that administer treats, pleasing sounds, positive reinforcements, etc. For example, a dog training collar that dispenses a pleasing sound, a pleasing odor or that utilizes a mild vibratory signal may be all that is needed for training the compliant animal.

The dog's intelligence will also come into play. A dog with more developed cognitive skills may only need to receive one or two electrical stimuli to be trained to avoid certain behaviors in the future, e.g., barking when the doorbell rings, running (or digging) out of the yard, failing to stay near the owner during walks, or defecating in the house. Dogs with a lower level of intelligence may need multiple types of negative stimuli to train against negative behaviors. These may include stronger shocks, more frequent shocks, supersonic noises, shrill noises, high-Hertz vibrations, and so forth.

It is proposed herein to employ a combination of a dog's determined intelligence with a dog's determined personality type to then identify an appropriate type of training device (or setting for a device) for an individual animal. It is further proposed herein to employ a combination of a dog's determined intelligence with a dog's determined personality type to then identify an appropriate dog training protocol for that specific dog. Still further, it is proposed herein to employ a combination of a dog's determined intelligence with a dog's determined personality type to then identify appropriate toys for entertaining or keeping the interest of the dog.

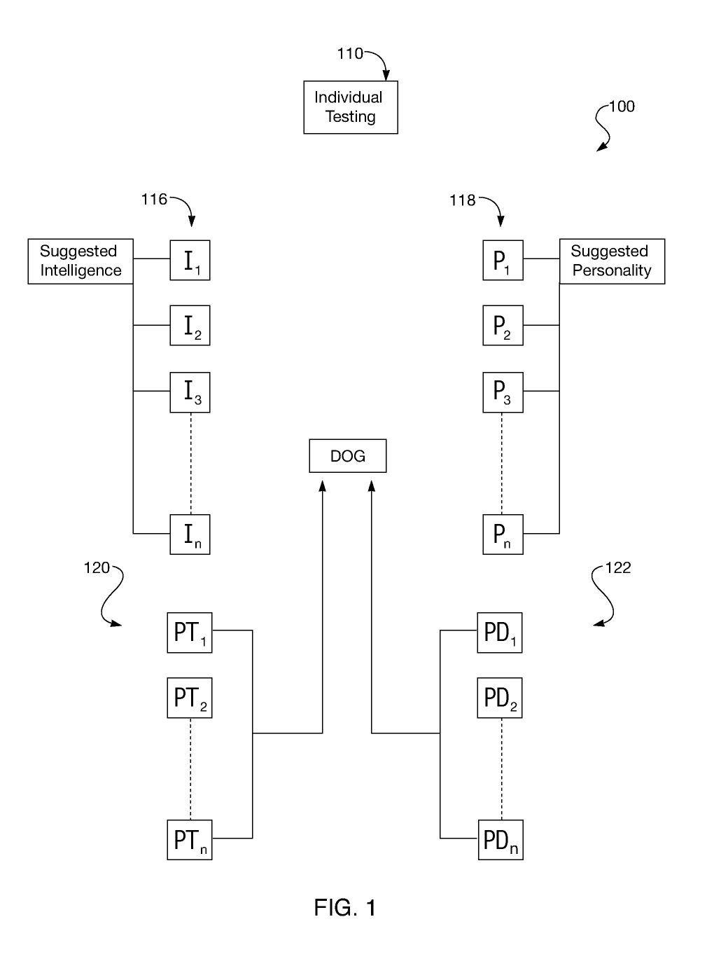

Under an embodiment herein, a dog owner or trainer may use testing assessments to select optimum combinations of training products and protocols. FIG. 1 shows a method 100 for training assessment and optimization. For the purpose of the example, assume that a trainer adopts such method 100 to develop and implement a training regimen for a dog. However, it should be noted that these procedures may also be adopted by an owner of a domesticated pet.

First, individual testing of a dog takes place. This is shown at Box 110. The trainer may perform the testing himself/herself, or may outsource the testing to a third party. Most preferably, the testing is conducted through the use of a guided series of inquiries offered by brochure or through on-line portal.

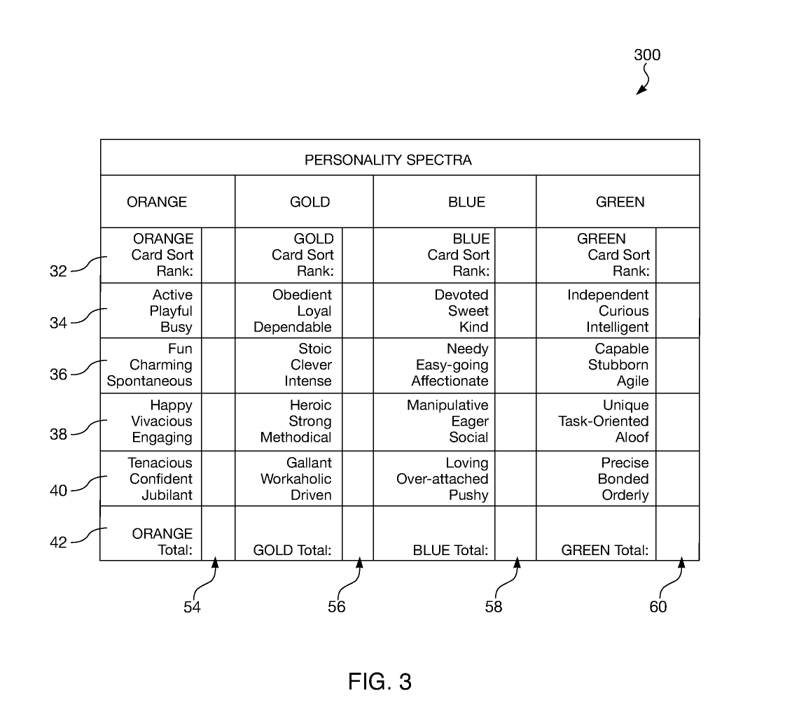

The testing 110 under one embodiment reveals an optimum learning style and areas of peak intelligence. In particular, the testing provides both intelligence testing and personality testing. The intelligence and personality assessments may comprise publicly available assessment tools, such as use of the Dognition™ protocol mentioned above and the use of tests such as the personality assessment shown in FIG. 3.

Step 110 comprises individual testing of the dog and subsequent review and evaluation of test data. As shown in FIG. 1, the test data will include both intelligence determination and personality assessment.

Box 116 schematically demonstrates a cognitive skill level assessment. The testing will identify a level of intelligence, depicted at I₁, I₂, . . . Iₙ, wherein each of I₁, I₂, . . . Iₙ represents a scaled range of intelligence. In one aspect, the scaled ranges I₁, I₂, . . . Iₙ are weighted according to the breed of the dog as determined as part of the testing in Box 110. Cognitive skill may also be judged based upon the extent of previous dog training the animal has experienced, or simply from individual, objective testing. In one aspect, a dog of a particular breed who is considered to be at the lower range of cognitive skill and who has never undergone obedience training will be at the lowest end of the intelligence range, e.g., I₁. At the other end, a dog of a particular breed who is considered to be at the higher range of cognitive skill and who has undergone extensive obedience training will be at the highest end of the intelligence range, e.g., Iₙ. In another aspect, intelligence of the dog is derived solely from individual testing to yield objective results.

Also as part of the method 100, individual testing 110 will include a personality assessment. Box 118 schematically demonstrates a personality assessment. The testing will identify a personality type of the animal, depicted at P₁, P₂, . . . Pₙ, wherein each of P₁, P₂, . . . Pₙ represents an assigned personality type. Personality types may be assigned, for example, as follows:

P₁ Active, playful and busy

P₂ Stoic, clever, intense

P₃ Loving, strongly attached, pushy

P₄ Precise, bonded orderly

P₅ Gallant, intense, driven

P₆ Obedient, loyal, dependable

P₇ Stubborn, strong

. . .

Pₙ Devoted, sweet, kind

In one aspect, the personality types P₁, P₂, . . . Pₙ are determined through mere observation and subjective evaluation by the pet owner or professional trainer. In another embodiment, the personality types P₁, P₂, . . . Pₙ are determined through formal testing conducted by the owner in response to questions or challenges presented to the owner (either through paper or through a web platform) and exercises given to the dog in response to the challenges. In still another aspect, the personality types P₁, P₂, . . . Pₙ are at least partially weighted based upon breed. In still another aspect, the personality types P₁, P₂, . . . Pₙ are determined through application of a personality matrix 300, such as that shown in FIG. 3.

In U.S. Patent Publ. No. 2011/0136086, a series of “cards” are employed. Each of the four cards can be characterized by a color and a corresponding graphic image on one side, and descriptive text upon the other side. The textual description presents an animal personality type. The accompanying textual description can expand upon the personality traits of the breed associated with, and thereafter designated by, that particular color.

In the arrangement of FIG. 3, a matrix 300 of personality types is provided to produce a color spectrum. In the “y” axis, characteristics 32, 34, 36, 38 and 40 are presented under each of colors orange, gold, blue and green. A numerical value is assigned to each characteristic, and then totaled at the bottom. Totals for each color are entered in boxes 54, 56, 58 and 60. The box 54, 56, 58, 60 with the highest score indicates a personality type for the animal. In the present invention, a similar matrix may be employed, with the results then being integrated into the assessment 116. Thus, for example, the following assignments may be made wherein the box with the highest score is listed first, and then in descending order:

| | |
|---|---|
| P₁ | Orange, gold, blue, green |
| P₂ | Gold, blue, green orange |
| P₃ | Blue, green, orange, gold |
| P₄ | Green, orange, gold, blue |
| P₅ | Orange, blue, green gold |
| P₆ | Blue, green, gold, orange |
| P₇ | Green, gold, orange, blue |
| . . . | |
| Pₙ | Green, gold, blue, orange |

Thus, personality types in this assessment are based upon a color spectrum.

Of course, it is understood that the color options presented herein are merely illustrative, and other color systems or non-color systems for assigning personality types P₁, P₂, . . . Pₙ 118 may be used to create useful spectra.

Under one embodiment of the method 100, the trainer uses the determined personality type 118 to select corresponding optimum training products PD₁, PD₂, . . . PDₙ for the dog. Training products are shown at 122. As one example, the personality testing may reveal that the dog has personality P₁. Under an embodiment, the personality P₁ suggests training product PD₁ as the optimal training tool for that particular dog. Similarly, personality P₂ suggests training product PD₂ is the optimal training tool for the particular dog.

It should be noted that the testing 110 may identify one personality type or a combination of such types. The identified personality type or types may indicate or suggest a single corresponding training product or a combination of training products.

As indicated above, the testing 100 also identifies levels of intelligence I₁, I₂, . . . Iₙ 116. Under one embodiment intelligence levels I₁, I₂, . . . Iₙ 116 represent an overall general ranking of the dog's intelligence. The trainer may then use an identified intelligence level I₁, I₂, . . . Iₙ to select one or more corresponding training protocols PT₁, PT₂, . . . , PTₙ.

Under one embodiment of the method 100, intelligence level I₁ suggests training protocol PT₁ as the optimal training protocol for that particular dog. Similarly, intelligence level I₂ suggests training protocol PT₂ is the optimal training tool for the particular dog.

It should be understood that intelligence levels I₁, I₂, . . . Iₙ 116 may represent different co-existing areas of peak intelligence. Under this alternative embodiment, testing may identify two or more areas of peak intelligence which may then suggest the use of one or more corresponding training protocols PT₁, PT₂, . . . PTₙ. Under an alternative embodiment, intelligence level I₁ may suggest a combination of training protocols PT₁ and PT₂ as the optimal training protocols for a particular dog.

In still another embodiment of the method 100, the trainer uses a novel combination of both the identified intelligence 116 and the determined personality type 118 to select a corresponding optimum training protocol 120 or a corresponding optimum training product 122 for the dog. For example, a dog who is at the higher end Iₙ of intelligence range 116 and who has a “Green, gold, orange, blue” P₄ personality profile may need training protocol PT₂ and developmental toy PD₃. A dog who is at a medium point I₄ of intelligence range 116 and who has a “Gold, blue, green orange” P₂ personality profile may need training protocol PT₃ and developmental toy PD₁. It is again observed that, for dogs, the cognitive skill level 116 may be somewhat tied to the dog's personality type 118. Thus, rather than using separate intelligence levels I₁, I₂, . . . Iₙ 116 and personality types P₁, P₂, . . . Pₙ 118, a single spectrum of dog type T₁, T₂, T₃, . . . Tₙ may be used to associate optimum training protocols PT₁, PT₂, . . . PTₙ or optimum training products PD₁, PD₂, . . . PDₙ for the dog.

In use, the method 100 described above may improve the training and learning experience of domesticated dogs (or other pets) by aligning the proper products and training protocols with their particular needs, abilities and breeding traits. Those with multiple pets have likely experienced the diversity in dog personality and intelligence. By utilizing research data and corresponding testing procedures, pet owners and trainers benefit by being guided as to what product and training protocol works best for a particular dog or type of dog. This approach saves, under one embodiment, time in training and money by avoiding the purchase of improper products. From the dog's point of view, this process streamlines the training process in the most efficient and humane way.

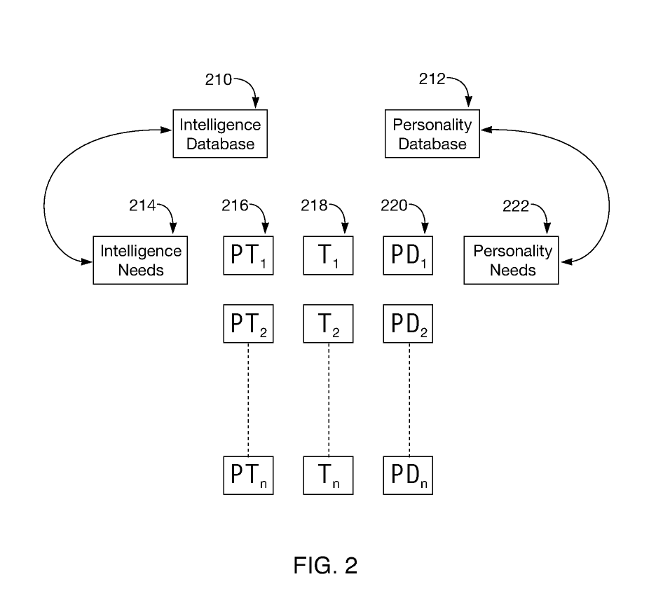

Researchers and trainers may use both personality and intelligence testing to build stereotypical personality and intelligence databases. FIG. 2 shows intelligence database 210 and personality database 212. Repeated intelligence testing across a population of dogs may generate database 210 suggesting certain intelligence needs 214, i.e. levels or types of intelligence that suggest certain optimal training protocols PT₁, PT₂, . . . PTₙ 216. Likewise, repeated personality testing across a population of dogs may generate database 212 suggesting certain personality needs 222, i.e. types of personality that suggest certain optimal training products PD₁, PD₂, . . . PDₙ 220.

As noted above, a combination of intelligence needs 214 and personality needs 222 may be used to suggest corresponding pet types T₁, T₂, . . . Tₙ, indicated at 218. Experimental application of product/protocol combinations to pet types T₁, T₂, . . . Tₙ may under an embodiment optimize product/protocol selection on an individual pet basis. As seen in FIG. 2, pet types T₁, T₂, . . . Tₙ 218 correspond to product and protocol combinations. As just one example, a trainer may test a dog for intelligence and personality type. In comparing the results to intelligence and personality database results 210, 212, the trainer identifies the dog as type T₂, which may represent a specific combination of intelligence needs 214 and personality needs 222. The trainer may then select protocol PT₂ and product PD₂ for optimum training of the dog based on the test results. Note that protocol PT₂ may represent one or more combination of protocols and that product PD₂ may represent one or more products.

The concept of intelligence 210 and personality 212 databases may include finding statistical associations between personality type and training products and between intelligence level and training protocols. The identified relationships may comprise associations between outcomes, e.g. time to learn a particular response, and predictors such as personality type, intelligence type, age, dog breed, environmental conditions or some combination thereof.

A statistical study under one embodiment may aggregate dogs of similar characteristics and then separate the population into two or more different testing groups. For example, study administrators may screen or assess a large number of dogs using a personality assessment tool. The study may identify a population of dogs that all rank high in a particular personality trait/characteristic, e.g. sociability. The study may wish to identify an optimal training product for teaching an animal to stay at one location upon command. (Of course it is determined that the study dogs have yet to learn the target behavior). Under one embodiment, a study protocol assigns to one group a collar device that applies negative stimuli, e.g., electrical shocks or high-Hz vibrations, while another group is assigned a collar device that only applies positive stimuli, e.g. pleasing tones or scents. The training products are then consistently applied within each group.

Under this embodiment, time to learn the target behavior or response may comprise an outcome variable. In other words, study administrators apply the training products to each group and measure the time each dog takes to learn the response, e.g., to learn the “stay” behavior. The outcome variable may be a continuous variable (e.g. number of aggregate hours to learn response) or simply binary (e.g., success or failure). Statistical associations between each product type and outcome may be determined using statistical methods such as analysis of variance or categorical data analysis but embodiments are not so limited. Additional predictors may be used alongside product groupings in order to control for additional variables such as age or breed of the animal. It should also be noted that analogous tools and methods may be used to associate intelligence levels with optimal training protocols.

As another approach, a study may recruit a large number of dogs for a study. Under one embodiment, administrators may assess the personality type of each dog using personality assessment tools. Under this embodiment, administrators also assess intelligence using intelligence assessment tools. In this manner, the personality type and intelligence level of each dog is known. Accordingly, a study protocol may then investigate various combinations of product type, product protocol, personality type and intelligence level. As one example, a study may administer a single product type for teaching a particular behavior to the entire study population. Time to learn the behavior may be measured as an outcome variable. Statistical methods may then be used to analyze collected data and infer which personality type may be best suited to the particular product.

This application of the proposed study design is analogous to the experiment suggested above and may also be used to associate intelligence level with optimal training protocols. However, this study design facilitates more sophisticated analysis options. For example, this study design may be used to investigate product type/training protocol combinations administered to (i) personality types, (ii) intelligence levels, and (iii) personality/intelligence combinations. This approach allows identification of optimal product/protocol regimens for a composite personality/intelligence type.

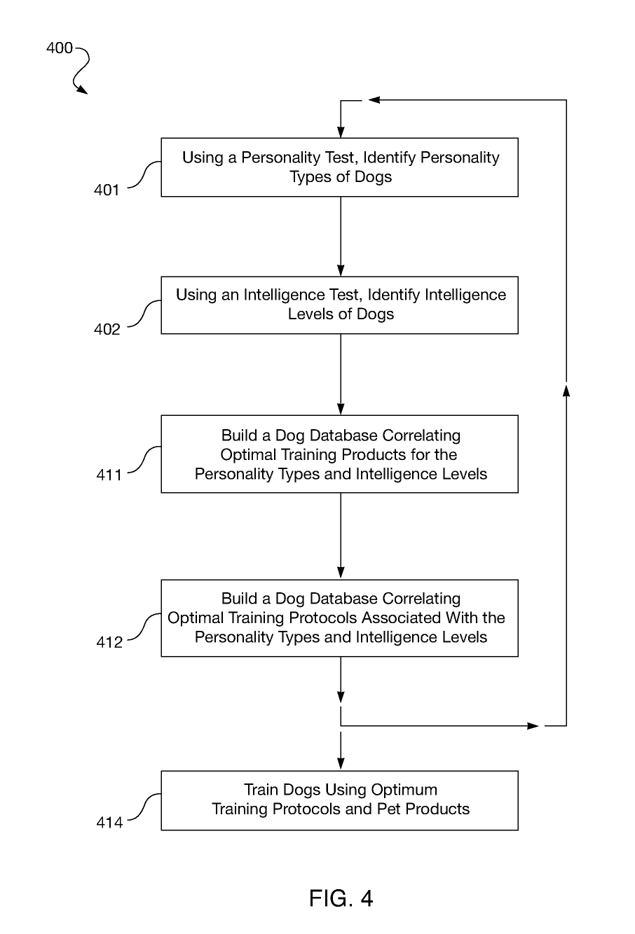

Based upon the above, a method 400 of building personality and intelligence databases is provided. FIG. 4 is a flowchart showing a method 400 for building and using dog databases to correlate optimum training products (PDOT) and optimum training protocols (IDOT).

First, personality types of dogs are determined. This is shown in Box 401. The personality types are determined by using personality tests, or assessments. Information about breeding and living environments may be incorporated.

In addition, intelligence levels of dogs are determined. This is provided in Box 402. The intelligence levels are determined by using intelligence tests, or assessments. Information about breeding and levels of training may be incorporated.

Also as part of the method 400, a dog database is built that correlates optimum training products for the various personality types and intelligence levels, in combination. This is indicated at Box 411.

Also as part of the method 400, a dog database is built that correlates optimum training protocols for the various personality types and intelligence levels, in combination. This is indicated at Box 412.

The steps 401, 402, 411 and 412 are repeated as part of building accurate databases.

As an optional step, the method 400 of FIG. 4 also includes training dogs using the optimum training protocols and pet products. This is provided at Box 414.

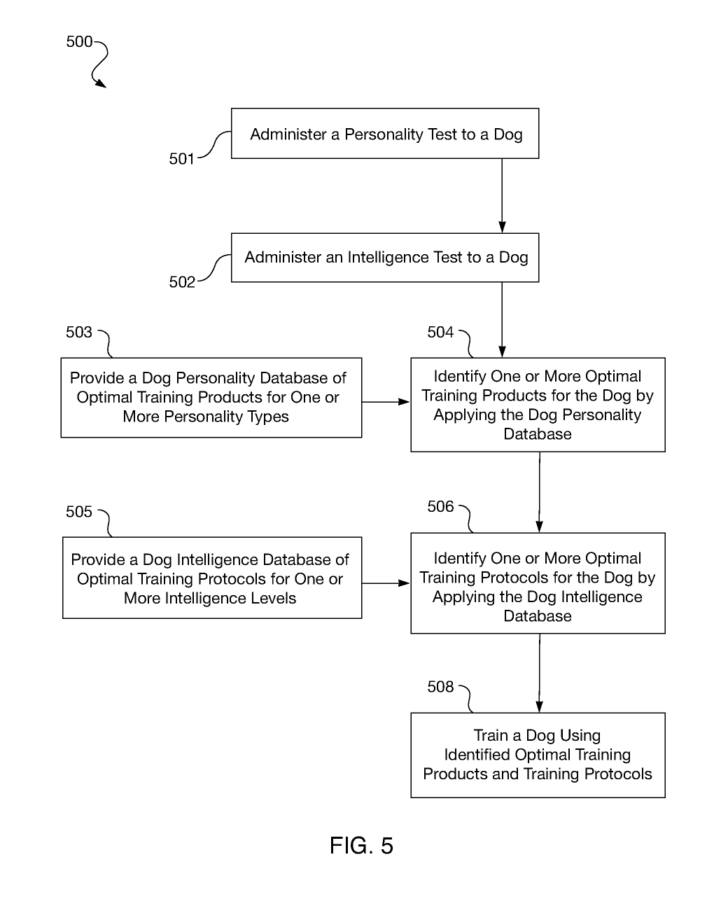

Also based upon the above, a method 500 is provided for selecting an optimal training product for a dog, or for selecting an optimal training protocol. FIG. 5 is a flowchart showing a method 500 for selecting optimal training products (PDOT) and optimal training protocols (IDOT) for a specific dog.

First, a personality test is administered to a dog. This is shown in Box 501. The personality test may be of any type, and may incorporate information about breeding, age and living environments.

In addition, an intelligence test is administered to the dog. This is provided in Box 502. The intelligence levels are determined by using intelligence tests, or assessments. Information about breeding and levels of training may be incorporated.

Next, a dog personality database is applied that correlates dog personality types with optimal training products. This is seen at Box 503. From this, one or more optimal training products is identified. This is indicated at Box 504.

In addition, a dog intelligence database is applied that correlates dog intelligence levels with optimal training protocols. This is seen at Box 505. From this, one or more optimal training protocols is identified. This is indicated at Box 506.

Preferably for the method 500, the databases of Boxes 503 and 505 are combined to generate the PDOT and IDOT of Boxes 504 and 506.

As an optional step, the method 500 of FIG. 5 also includes training dogs using the identified optimal training products and training protocols. This is provided at Box 508.

Additionally based upon the above, a method 600 is provided for testing a dog. The method 600 first includes testing a dog to determine a personality profile. This is seen at Box 601. Determining a personality profile 601 may include (i) observing the dog in connection with certain daily activities, (ii) empirically testing the dog by using exercises or commands, (iii) assessing breed type of the dog, (iv) determining a living environment of the dog, or (v) combinations thereof. The method 600 further includes testing the dog for determining a cognitive skill level. This is provided at Box 602. Determining a cognitive skill level 602 may include (i) assessing a breed type of the dog, (ii) identifying a degree of training through which the dog has been taken, (iii) empirically testing the dog by using exercises or commands, (iv) combinations thereof. In one aspect, the steps of Boxes 601 and 602 are merged into one test. For example, determining a cognitive skill level may include (v) determining a personality type, from a set of optional personality types, for the dog.

The method 600 also includes identifying an optimum training protocol for the dog. This is shown at Box 603. The method 600 additionally includes identifying an optimum pet toy for the dog (seen at Box 604), and identifying an optimum developmental product for the dog (seen at Box 605). The steps of Boxes 603, 604 and 605 are based upon the determined personality profile and intelligence range. Further determining combinations of 603, 604, and 605 is provided at Box 606 based upon the determined personality profile and intelligence range.

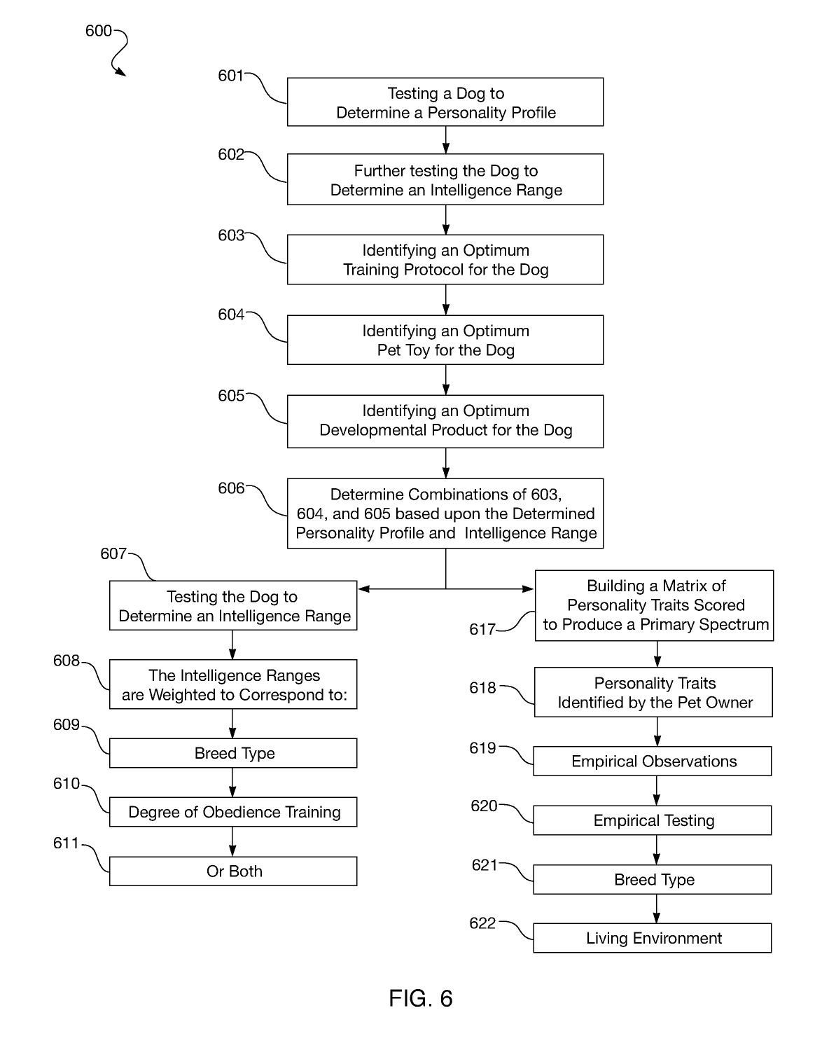

With continuing reference to FIG. 6, steps 607 for testing a dog to determine an intelligence range is shown wherein intelligence ranges are weighted 608 to correspond to breed type 609, degree of obedience training 610, or both 611. Furthermore, steps 617 for building a matrix of personality traits scored to produce a primary spectrum is shown wherein the personality traits are identified by the pet owner 618, along with empirical observations 619, empirical testing 620, breed type 621 and, optionally, living environment 622.

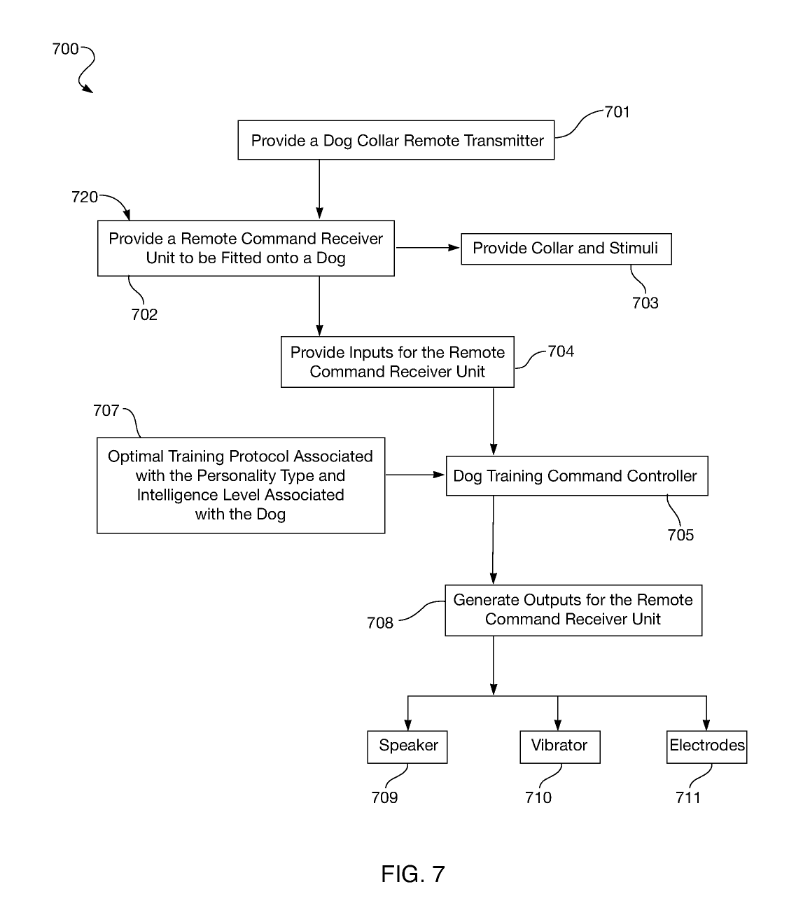

For some training goals such as keeping a dog within an “Invisible Fence”® area or for bark reduction, certain identified training protocols, preferably an optimal one, may be embedded in a dog collar or device to provide the ability to train the dog autonomously by the dog collar itself as well as through key commands from a remote dog collar transmitter or device. FIG. 7 presents a flowchart for a method 700 for operating, or for tuning, a dog collar training system for a canine. The system will include a dog collar remote transmitter and a remote command receiver unit. The remote command receiver unit will be supported on the dog by a collar or suitable harness, as desired.

The method 700 first includes providing a dog collar remote transmitter 701. This is shown at Box 701. The transmitter sends signals to a receiver unit via Bluetooth, IR, Zigbee or other wireless protocol.

The method 700 also includes providing a remote command receiver unit. This is seen at Box 702. The receiver unit is part of the dog collar training system 720, and receives signals from the dog collar remote transmitter 701.

The dog collar preferably includes a collar, and is configured with stimuli, as shown in Box 703. The remote command receiver unit is then fitted onto a dog.

The method 700 includes transmitting data commands, or signals, from the remote transmitter 701 to the remote command receiver 702. This is shown at Box 704. The transmitted data commands are received as inputs 704 to a dog training command controller, seen at Box 705.

The commands are processed by the dog training command controller 705. Various levels are sent through the outputs block 708 for actuation by a speaker 709, a vibrator 710, or a set of shocking electrodes 711, resulting in various stimuli to the dog. Some commands result in positive stimuli to the dog including a pleasing sound, a pleasing odor or a mild vibratory signal while some commands result in negative stimuli to the dog including harsh or loud sounds, intense vibrations, or a high level of electric shock.

With continuing reference to FIG. 7, the dog collar 720 may receive voice over data signals or audio at the remote command receiver 702 wherein voice commands from the dog trainer are passed through to the speaker 709 for reproduction into the dog's ears. The voice over data commands provide the dog trainer with the advantage of speaking to the dog at a low or normal sound level through the speaker 709 regardless of how far the dog has wandered. This eliminates the occasional need to yell at the dog due to distance and therefore reduces the irritation to others in hearing range of the training area. Further, this allows the dog trainer to whisper into the dog's ears and gives the appearance that the dog is behaving independently but is actually being commanded by the dog trainer.

With continuing reference to FIG. 7, some training goals associated with staying inside an invisible fence or barking reduction, the dog collar 720 may perform as the optimum development product 605 and receive commands to operate autonomously based on the dog training controller 705 using an optimal training protocol associated with the intelligence type of the subject dog 707. Alternately, autonomous operation of the dog collar 720 may include a series of increasing levels of various positive stimuli until a training goal is achieved. Further, autonomous operation of the dog collar 720 may include a series of increasing levels of various negative stimuli until a training goal is achieved. Furthermore, autonomous operation of the dog collar 720 may include a series of trial-and-error stimuli and measured responses.

A microphone in the dog collar 720 may sense barking or other dog sounds such as whining or whimpering. The dog training command controller 705 may sense or interpret the various dog sounds and vary the stimuli based on what the dog is saying in dog sounds. Further, the dog training command controller 705 may include a human speech recognition system whereby the dog trainer's verbal commands such as a pleasing low level vibration to the dog collar may be verbal as an alternative to key commands from the dog collar remote transmitter 701. The speaker 709 volume level is calibrated in decibels such that remote verbal commands coming from the speaker are set at approximately the same level as if the dog is standing nearby the dog trainer. For pleasant sounds in positive stimuli, the volume level is set at approximately the same level as verbal commands whereas the strong negative stimuli sounds are calibrated to be at a much higher level such as 80 db. Alternatively, speaker 709 or an auxiliary ultrasonic speaker may be capable of ultrasonic sound emissions and would be calibrated to just under a dog's threshold of pain for negative stimuli. Verbal commands may be stored digitally within the dog training command controller 705 for generating verbal commands such as “LEAVE IT” or “GOOD DOG.”

It is understood that the systems and methods of FIGS. 1 and 2 and FIG. 4 through 7 for training dogs are merely illustrative. Other arrangements may be employed in accordance the embodiments set forth below. Further, variations of the systems and methods described herein may comply with the spirit of the embodiments set forth herein.

I claim:

1. A method of developing a dog, comprising:
testing a dog to determine a profile; and
(i) identifying an optimum training protocol for the dog, (ii) identifying an optimum developmental product for the dog, (iii) identifying an optimum pet toy for the dog, or (iv) combinations thereof, based upon the determined profile.

2. The method of claim 1, wherein the profile is based upon a (i) personality of the dog, (ii) a cognitive skill level of the dog, or (iii) both.

3. The method of claim 2, wherein testing the dog comprises determining a personality profile.

4. The method of claim 3, wherein the personality profile comprises a matrix of personality traits which are scored to produce a primary spectrum.

5. The method of claim 4, wherein the personality traits are identified by a pet owner based upon (i) observations of the dog, (ii) empirical testing of the dog, (iii) breed type of the dog, (iv) living environment, or (v) combinations thereof.

6. The method of claim 2, wherein testing the dog comprises determining a cognitive skill level of the dog.

7. The method of claim 6, wherein:
testing the dog to determine a cognitive skill level comprises selecting an intelligence range from a plurality of intelligence ranges; and
the intelligence ranges are weighted to correspond to (i) breed type, (ii) degree of obedience training, or (iii) both, for the dog.

8. The method of claim 6, wherein the cognitive skill level of the dog considers (i) breed type of the dog, (ii) degree of previous obedience training received by the dog, (iii) personality, or (iv) combinations thereof.

9. The method of claim 1, further comprising:
training the dog (i) based on the identified optimum training protocol, (ii) using the identified optimum developmental product, or (iii) both.

10. The method of claim 1, further comprising:
purchasing an optimum pet toy for the dog based upon an identified (i) personality of the dog, (ii) cognitive skill level of the dog, or (iii) combination thereof.

11. A system for training a dog, comprising:
a test to be used for identifying a profile of a dog, wherein the profile is selected from a group of profiles;
a products database including information of optimal training products for each of the profiles in the group of profiles, wherein the optimal training product information associates a profile with one or more corresponding optimal training products; and
a training database including information of optimal training protocols for each of the profiles in the group of profiles, wherein the optimal training protocol information associates a profile with one or more corresponding training protocols.

12. The system of claim 11, wherein the profiles have been derived from (i) observations of dogs, (ii) empirical testing of dogs while the dogs perform exercises, (iii) considering breed types for the dogs, and (iv) combinations thereof.

13. The system of claim 11, wherein the test comprises (i) a personality test for identifying at least one personality type of a dog, (ii) an intelligence test for identifying at least one intelligence level of the dog; or (iii) both.

14. The system of claim 13, wherein the personality test and the intelligence test are combined into a single test such that:
optimal training product information associates the assessed personality type combined with the assessed intelligence level with one or more corresponding optimal training products; and
optimal training protocol information associates the assessed personality type combined with the assessed intelligence level with one or more corresponding training protocols

15. The system of claim 13, wherein:
the products database includes information of optimal training products for each of the at least one personality type and for each of the at least one intelligence level, wherein the optimal training product information associates a personality type combined with an intelligence level with one or more corresponding optimal training products; and
the training database includes information of optimal training protocols for each of the at least one personality type and for each of the at least one intelligence level, wherein the optimal training protocol information associates a personality type combined with an intelligence level with one or more corresponding training protocols.

16. The system of claim 15, wherein associating a personality type combined with an intelligence level with one or more corresponding optimal training products is derived from determining the one or more training products that maximize efficiency of the dog in learning one or more new behaviors.

17. The system of claim 15, wherein associating a personality type combined with an intelligence level with one or more corresponding training protocols is derived from determining the one or more training protocols that maximize efficiency of the dog in learning one or more new behaviors.

18. The system of claim 15, wherein associating the personality type combined with an intelligence level with one or more corresponding training protocols and with one or more corresponding training products is derived from administering the personality test to each dog of a population of dogs, and identifying each dog's personality type based on the personality test.

19. The system of claim 15, wherein associating the personality type combined with an intelligence level with one or more corresponding training protocols and with one or more corresponding training products is derived from administering the intelligence test to each dog of the population of dogs, and identifying each dog's intelligence type based on the intelligence test.

20. The system of claim 15, wherein associating the personality type combined with an intelligence level with one or more corresponding training protocols and with one or more corresponding training products is derived from (i) finding statistical associations between personality types and training products, (ii) finding statistical associations between intelligence levels and training products, (iii) finding statistical associations between intelligence levels and training protocols, and (iv) finding statistical associations between personality types and training protocols.

21. The system of claim 20, wherein associating the personality type combined with an intelligence level with one or more corresponding training protocols and with one or more training products is derived from applying at least one of the one or more training products and the one or more training protocols to each dog of the population of dogs to teach a behavior of the one or more new behaviors.

22. The system of claim 21, wherein associating the personality type combined with an intelligence level with one or more corresponding training protocols and with one or more training products comprises determining time to learn the new behaviors.

23. The system of claim 15, wherein associating the personality type combined with an intelligence level with one or more corresponding training protocols and with one or more training products is derived from using at least one statistical inference test to evaluate an effect of combinations of the at least one personality type, the at least one intelligence type, the one or more training products and the one or more training protocols on outcome data.

24. A method comprising:
identifying at least one personality type of a dog using a personality test;
identifying at least one intelligence level of a dog using an intelligence test;
building a personality database including information of optimal training protocols and training products for each type of the at least one personality type;
building an intelligence database including information of optimal training protocols and training products for each type of the at least one intelligence type;
administering the personality test to a dog to identify the dog's personality type of the at least one personality type;
administering the intelligence test to the dog to identify the dog's intelligence type of the at least one intelligence type; and
based on the personality database information and intelligence database information, associating one or more optimum training protocols for the dog associated with the identified personality type, the identified intelligence type, or both; and
based on the personality database information and intelligence database information, associating one or more optimum training products for the dog associated with the identified personality type, the identified intelligence type, or both.

25. The method of claim 24, wherein the personality test comprises a matrix of personality traits which are scored to produce a primary spectrum.

26. The method of claim 25, wherein the personality traits are identified by a pet owner based upon (i) empirical observations of the dog, (ii) empirical testing of the dog, (iii) breed type of the dog, (iv) living environment, or (v) combinations thereof.

27. The method of claim 24, wherein:
administering the intelligence test comprises selecting an intelligence type from a plurality of intelligence types; and
the intelligence types are weighted to correspond to (i) breed type, (ii) degree of obedience training, or (iii) both, for the dog.

28. The method of claim 24, administering the intelligence test comprises observing the dog's performance of a plurality of exercises designed for testing intelligence, and recording data of the performance.

29. The method of claim 24, wherein the intelligence test comprises observing the dog's performance of a plurality of exercises designed for testing intelligence, and recording data of the performance.
